(19) United States
(12) Patent Application Publication
(10) Pub. No.: US 2026/0314995 A1
(43) Pub. Date: Oct. 15, 2026

(54) ELECTRICAL IMPEDANCE MYOGRAPHY FOR THE EARLY DETECTION OF COMPARTMENT SYNDROME

(71) Applicant: Beth Israel Deaconess Medical Center, Inc., Boston, MA (US)

(72) Inventors: Seward B. Rutkove, Brookline, MA (US); Ara Nazarian, Watertown, MA (US); John Wixted, Boston, MA (US)

(73) Assignee: Beth Israel Deaconess Medical Center, Inc., Boston, MA (US)

(21) Appl. No.: 19/476,553

(22) PCT Filed: Apr. 18, 2024
(86) PCT No.: PCT/US2024/025271

§ 371 Date: Oct. 17, 2025

Related U.S. Application Data
(60) Provisional application No. 63/496,788, filed on Apr. 18, 2023.

(51) Int. Cl. A61B 5/00; A61B 5/0537

(52) U.S. Cl. CPC A61B 5/4519; A61B 5/0537; A61B 5/6887; A61B 5/7264; A61B 5/7271

(57) ABSTRACT

A method and apparatus for detecting compartment syndrome. The method includes passing, using a first pair of electrodes, a current through a region of muscle of a user; measuring, using a second pair of electrodes, one or more surface voltages; calculating, based on the one or more surface voltages, one or more measurements over a span of time; and determining, based on the one or more measurements over the span of time, whether the user is experiencing early muscle ischemia.

### TECHNICAL FIELD

The techniques described herein relate generally to early detection, identification, and quantification of compartment syndrome and measuring, calculating and presenting an ischemia injury index.

### BACKGROUND

Compartment syndrome is an orthopedic condition caused by pressure buildup in one of the body's many compartments. There are two major types of compartment syndrome: acute compartment syndrome (ACS) and chronic compartment syndrome (CCS).

Acute Compartment Syndrome (ACS) is one of the most devastating orthopedic conditions, affecting any one of the body's many compartments. It is typically caused by injury, and treated with a fasciotomy (surgical procedure involving cutting open fascia). If sufficiently severe, ACS may result in disability and amputation. Diagnosing limbs at risk for ACS before the occurrence of irreversible damage to muscle and nerve is critical. Standard approaches for the diagnosis of impending compartment syndrome include clinical evaluation of the limb, such as assessment for “tightness” of overlying skin, reduced pulses distally and degree of pain; none of which are specific or sensitive.

Chronic Compartment Syndrome (CCS) is typically less severe than ACS and develops over a longer period of time. CCS typically results from exercise involving repeated stress or strain on a muscle or compartment. It can be treated by cessation of the exercise that causes the repeated stress or strain.

### SUMMARY

The embodiments of the technology described in this application can be used to detect either ACS or CCS.

Methods: We evaluated the ability of EIM to detect early muscle ischemia in an established murine model of compression-induced muscle injury, where we collected resistance, reactance, and their dimensionless product, defined as Relative Injury Index (RII) during the study.

As a result, our model generated reproducible hypoxia, confirmed by Hypoxyprobe™ staining of endothelial regions within muscle. Under conditions of ischemia, we demonstrated a reproducible, stable and significant escalation in resistance, reactance and RII values, when compared to uninjured control limbs. This data illustrates that EIM can be used to detect ACS, and also more indolent forms (chronic compartment syndrome (CCS)).

The inventors have developed a novel method to detect ACS, and by extension CCS, via the application of electrical impedance myography (EIM), where a weak, high frequency alternating current is passed between one pair of electrodes through a region of tissue and the resulting surface voltages measured via a second pair. An example of a weak current in accordance with embodiments of the disclosed technology is a current with a root mean square (RMS) of less than 1 milliampere (mA). An example of a high frequency current in accordance with embodiments of the disclosed technology is a current with a frequency in the range of 1 kilohertz (kHz) to 10 megahertz (MHz). EIM may be used for biometric purposes. For example, increase in muscle strength or fitness or mass may be measured by means of EIM and periods of time may be required for increase in muscular strength.

An increase (or decrease) in muscle strength due to growth or shrinkage or injury of the muscle requires months. EIM measurements to measure growth or shrinkage of a muscle or muscle group involve measurements over a period of months.

Some embodiments are directed to a method for detecting compartment syndrome, the method comprising: passing, using a first pair of electrodes, a current through a region of muscle of a user; measuring, using a second pair of electrodes, one or more surface voltages; calculating, based on the one or more surface voltages, one or more measurements over a span of time; and determining, based on the one or more measurements over the span of time, whether the user is experiencing early muscle ischemia.

In some embodiments, the determining, based on the one or more measurements over the span of time, whether the user is experiencing early muscle ischemia comprises comparing the one or more measurements to threshold values.

In some embodiments, the current is an alternating current.

In some embodiments, the current is a weak current.

In some embodiments, the current is a high frequency current.

In some embodiments, the current is a directionally specific current.

In some embodiments, the one or more measurements comprise a resistance.

In some embodiments, the one or more measurements comprise a reactance.

In some embodiments, the one or more measurements comprise a product of a resistance and reactance.

In some embodiments, the one or more measurements comprise a product of a resistance and reactance divided by a product of a baseline resistance and baseline reactance.

In some embodiments, the method further comprises determining whether the user has compartment syndrome.

In some embodiments, the first pair of electrodes and the second pair of electrodes are all positioned beneath a cast.

In some embodiments, passing, using the first pair of electrodes, a current through a region of muscle of a user comprises varying a frequency of the alternating current over a plurality of frequency values. In some embodiments, measuring, using the second pair of electrodes, one or more surface voltages comprises measuring a plurality of voltages from the second pair of electrodes, each voltage of the plurality of voltages corresponding to a frequency value of the plurality of frequency values. In some embodiments, calculating, based on the one or more surface voltages, one or more measurements over a span of time comprises calculating a plurality of measurements, each measurement of the plurality of measurements corresponding to a voltage of the plurality of voltages. In some embodiments, determining, based on the one or more measurements over the span of time, whether the user is experiencing early muscle ischemia comprises determining whether the user is experiencing early muscle ischemia based on the plurality of frequency values and the plurality of measurements.

In some embodiments, determining whether the user is experiencing early muscle ischemia based on the plurality of frequency values and the plurality of measurements comprises determining a frequency-averaged parameter for at least a portion of the plurality of frequency values.

In some embodiments, determining whether the user is experiencing early muscle ischemia based on the plurality of frequency values and the plurality of measurements comprises determining whether the frequency-averaged parameter exceeds a threshold value.

In some embodiments, passing, using a first pair of electrodes, a current through a region of muscle of a user comprises passing a current through a region of muscle of a user along each axis of a plurality of axes. In some embodiments, measuring, using the second pair of electrodes, one or more surface voltages comprises measuring a plurality of voltages from the second pair of electrodes, each voltage of the plurality of voltages corresponding to an axis of the plurality of axes. In some embodiments, calculating, based on the one or more surface voltages, one or more measurements over a span of time comprises calculating a plurality of measurements, each measurement of the plurality of measurements corresponding to a voltage of the plurality of voltages. In some embodiments, determining, based on the one or more measurements over the span of time, whether the user is experiencing early muscle ischemia comprises determining whether the user is experiencing early muscle ischemia based on the plurality of axes and the plurality of measurements.

In some embodiments, the method further comprises obtaining a pressure measurement for the region of muscle, and the determining, based on the one or more measurements over the span of time, whether the user is experiencing early muscle ischemia comprises determining, based on the one or more measurements over the span of time and the pressure measurement, whether the user is experiencing early muscle ischemia.

In some embodiments, the method further comprises treating the early muscle ischemia by releasing pressure in the region of muscle by creating an incision into the region of muscle.

In some embodiments, the method further comprises treating the early muscle ischemia by instructing the user to cease engagement in an activity causing the early muscle ischemia.

Some embodiments are directed to a system for detecting compartment syndrome, the system comprising: a first pair of electrodes; a second pair of electrodes; and at least one computer communicatively coupled to the first pair of electrodes and the second pair of electrodes, the at least one computer comprising a computer processor configured to perform the acts of passing, using the first pair of electrodes, a current through a region of muscle of a user; measuring, using the second pair of electrodes, one or more surface voltages; calculating, based on the one or more surface voltages, one or more measurements over a span of time; and determining, based on the one or more measurements over the span of time, whether the user is experiencing early muscle ischemia.

In some embodiments of the system, the determining, based on the one or more measurements over the span of time, whether the user is experiencing early muscle ischemia comprises comparing the one or more measurements to threshold values.

In some embodiments of the system, the current is an alternating current.

In some embodiments of the system, the current is a weak current.

In some embodiments of the system, the current is a high frequency current.

In some embodiments of the system, the current is a directionally specific current.

In some embodiments of the system, the one or more measurements comprise a resistance.

In some embodiments of the system, the one or more measurements comprise a reactance.

In some embodiments of the system, the one or more measurements comprise a product of a resistance and reactance.

In some embodiments of the system, the one or more measurements comprise a product of a resistance and reactance divided by a product of a baseline resistance and baseline reactance.

In some embodiments of the system, the computer processor is further configured to perform the act of determining whether the user has compartment syndrome.

In some embodiments of the system, the first pair of electrodes and the second pair of electrodes are positioned beneath the surface of a cast.

In some embodiments of the system, passing, using the first pair of electrodes, a current through a region of muscle of a user comprises varying a frequency of the alternating current over a plurality of frequency values. In some embodiments of the system, measuring, using the second pair of electrodes, one or more surface voltages comprises measuring a plurality of voltages from the second pair of electrodes, each voltage of the plurality of voltages corresponding to a frequency value of the plurality of frequency values. In some embodiments of the system, calculating, based on the one or more surface voltages, one or more measurements over a span of time comprises calculating a plurality of measurements, each measurement of the plurality of measurements corresponding to a voltage of the plurality of voltages. In some embodiments of the system, determining, based on the one or more measurements over the span of time, whether the user is experiencing early muscle ischemia comprises determining whether the user is experiencing early muscle ischemia based on the plurality of frequency values and the plurality of measurements.

In some embodiments of the system, determining whether the user is experiencing early muscle ischemia based on the plurality of frequency values and the plurality of measurements comprises determining a frequency-averaged parameter for at least a portion of the plurality of frequency values.

In some embodiments of the system, determining whether the user is experiencing early muscle ischemia based on the plurality of frequency values and the plurality of measurements comprises determining whether the frequency-averaged parameter exceeds a threshold value.

In some embodiments of the system, passing, using a first pair of electrodes, a current through a region of muscle of a user comprises passing a current through a region of muscle of a user along each axis of a plurality of axes. In some embodiments of the system, measuring, using the second pair of electrodes, one or more surface voltages comprises measuring a plurality of voltages from the second pair of electrodes, each voltage of the plurality of voltages corresponding to an axis of the plurality of axes. In some embodiments of the system, calculating, based on the one or more surface voltages, one or more measurements over a span of time comprises calculating a plurality of measurements, each measurement of the plurality of measurements corresponding to a voltage of the plurality of voltages. In some embodiments of the system, determining, based on the one or more measurements over the span of time, whether the user is experiencing early muscle ischemia comprises determining whether the user is experiencing early muscle ischemia based on the plurality of axes and the plurality of measurements.

In some embodiments, the system further comprises a hand-held apparatus, wherein the first pair of electrodes and the second pair of electrodes are mounted on the hand-held apparatus; and the first pair of electrodes and the second pair of electrodes are communicatively coupled to the at least one computer by means of the hand-held apparatus.

Some embodiments are directed to at least one non-transitory computer-readable storage medium storing processor-executable instructions that, when executed by at least one computer hardware processor, cause the at least one computer hardware processor to perform passing, using a first pair of electrodes, a current through a region of muscle of a user; measuring, using a second pair of electrodes, one or more surface voltages; calculating, based on the one or more surface voltages, one or more measurements over a span of time; and determining, based on the one or more measurements over the span of time, whether the user is experiencing early muscle ischemia.

In some embodiments of the at least one non-transitory computer-readable storage medium, the determining, based on the one or more measurements over the span of time, whether the user is experiencing early muscle ischemia comprises comparing the one or more measurements to threshold values.

In some embodiments of the at least one non-transitory computer-readable storage medium, the current is an alternating current.

In some embodiments of the at least one non-transitory computer-readable storage medium, the current is a weak current.

In some embodiments of the at least one non-transitory computer-readable storage medium, the current is a high frequency current.

In some embodiments of the at least one non-transitory computer-readable storage medium, the current is a directionally specific current.

In some embodiments of the at least one non-transitory computer-readable storage medium, the one or more measurements comprise a resistance.

In some embodiments of the at least one non-transitory computer-readable storage medium, the one or more measurements comprise a reactance.

In some embodiments of the at least one non-transitory computer-readable storage medium, the one or more measurements comprise a product of a resistance and reactance.

In some embodiments of the at least one non-transitory computer-readable storage medium, the one or more measurements comprise a product of a resistance and reactance divided by a product of a baseline resistance and baseline reactance.

In some embodiments of the at least one non-transitory computer-readable storage medium, the computer processor is further configured to perform the act of determining whether the user has compartment syndrome.

In some embodiments of the at least one non-transitory computer-readable storage medium, the first pair of electrodes and the second pair of electrodes are positioned beneath the surface of a cast.

In some embodiments of the at least one non-transitory computer-readable storage medium, passing, using the first pair of electrodes, a current through a region of muscle of a user comprises varying a frequency of the alternating current over a plurality of frequency values. In some embodiments of the at least one non-transitory computer-readable storage medium, measuring, using the second pair of electrodes, one or more surface voltages comprises measuring a plurality of voltages from the second pair of electrodes, each voltage of the plurality of voltages corresponding to a frequency value of the plurality of frequency values. In some embodiments of the at least one non-transitory computer-readable storage medium, calculating, based on the one or more surface voltages, one or more measurements over a span of time comprises calculating a plurality of measurements, each measurement of the plurality of measurements corresponding to a voltage of the plurality of voltages. In some embodiments of the at least one non-transitory computer-readable storage medium, determining, based on the one or more measurements over the span of time, whether the user is experiencing early muscle ischemia comprises determining whether the user is experiencing early muscle ischemia based on the plurality of frequency values and the plurality of measurements.

In some embodiments of the at least one non-transitory computer-readable storage medium, determining whether the user is experiencing early muscle ischemia based on the plurality of frequency values and the plurality of measurements comprises determining a frequency-averaged parameter for at least a portion of the plurality of frequency values.

In some embodiments of the at least one non-transitory computer-readable storage medium, determining whether the user is experiencing early muscle ischemia based on the plurality of frequency values and the plurality of measurements comprises determining whether the frequency-averaged parameter exceeds a threshold value.

In some embodiments of the at least one non-transitory computer-readable storage medium, passing, using a first pair of electrodes, a current through a region of muscle of a user comprises passing a current through a region of muscle of a user along each axis of a plurality of axes. In some embodiments of the at least one non-transitory computer-readable storage medium, measuring, using the second pair of electrodes, one or more surface voltages comprises measuring a plurality of voltages from the second pair of electrodes, each voltage of the plurality of voltages corresponding to an axis of the plurality of axes. In some embodiments of the at least one non-transitory computer-readable storage medium, calculating, based on the one or more surface voltages, one or more measurements over a span of time comprises calculating a plurality of measurements, each measurement of the plurality of measurements corresponding to a voltage of the plurality of voltages. In some embodiments of the at least one non-transitory computer-readable storage medium, determining, based on the one or more measurements over the span of time, whether the user is experiencing early muscle ischemia comprises determining whether the user is experiencing early muscle ischemia based on the plurality of axes and the plurality of measurements.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows an experimental set up for ischemia induction and EIM measurement in the right hind limb. FIG. 1A shows electrodes attached to skin over the anterolateral muscle compartment, after the skin is shaved. FIG. 1B shows a size 3 neonatal blood pressure cuff placed around the leg. FIG. 1C shows an elastic rubber band is tightened around the upper thigh and the blood pressure cuff is inflated to 300 mmHg.

FIG. 2 shows evidence of ischemic injury as evidenced by collagen VI staining (green 200) and Hypoxyprobe™ probe staining (red 202 and 203) in the tibialis anterior muscle of the ACS injured right hind limb (right panel) and the lack of Hypoxyprobe™ probe staining (red 202 and 203) in the uninjured left hind limb (left panel) from the same animal. The bar shown represents 50 microns.

FIG. 3A shows resistance, FIG. 3B shows reactance, and FIG. 3C shows Relative Injury Index (RII) changes over time with progressive ischemia in a representative compartment syndrome rat model. Both impedance features and the RII indicate consistent multifrequency changes in rat muscle. The time stamps for graphs show Baseline and Inflated tourniquet (bottom two lines), followed by ischemia times from 2 min to 40 min.

FIG. 4 shows average reactance, resistance and Relative Injury Index (RII) values for rats. Significant reactance values compared to 0 (baseline tourniquet inflation), 0.046, 0.046, 0.03 and 0.02. Single asterisk “*” signifies significant resistance values compared to 0 (baseline tourniquet inflation), 0.031, and 0.032. Double asterisk signifies significant RII values compared to 0 (baseline tourniquet inflation), 0.007, 0.032, and 0.048.

FIG. 5A shows the RII over time for ischemia (n=12) and control (n=5) groups of animals.

FIG. 5B shows the change over time in resistance, reactance, phase angle, and intracompartmental pressure (ICP) measurements in the limb of a single pig in which muscle ischemia was induced.

FIG. 6 is a diagram illustrating an example of a device that performs electrical impedance myography, according to some embodiments.

FIG. 7 is a flow chart illustrating a method for detecting compartment syndrome, according to some embodiments.

FIG. 8 is a flow chart illustrating a multi-frequency method for detecting compartment syndrome, according to some embodiments.

FIGS. 9A-C show plots of measured electrical parameters versus frequency, using multi-frequency electrical impedance myography obtained in accordance with various aspects of some embodiments.

FIG. 10 is a diagram illustrating an example of a device that performs rotational electrical impedance myography, according to some embodiments.

FIG. 11 is a diagram illustrating an example of a device that performs electrical impedance myography at a plurality of angles, according to some embodiments.

FIG. 12 is a flow chart illustrating a method for performing rotational electrical impedance myography, according to some embodiments.

FIG. 13 is a diagram illustrating an example of a hand-held device that performs electrical impedance myography, according to some embodiments.

FIG. 14 shows, schematically, an illustrative computer on which any aspect of the present disclosure may be implemented.

### DETAILED DESCRIPTION

Compartment Syndrome remains one of the most devastating of orthopedic conditions. Individuals may contract either Acute Compartment Syndrome (ACS) or Chronic Compartment Syndrome (CCS). With an average incidence of approximately 7.3/100,000 males and 0.7/100,000 females, full-blown compartment syndrome can result in disability, which, if sufficiently severe, may warrant amputation. The condition can affect any one of the body's many compartments-regions with fixed volumes, anatomically separated from other areas by inflexible fascial planes. The most common location for acute compartment syndrome is the anterior tibial, usually resulting from a tibial fracture or crush injury, though a number of other regions can also be affected, including but not limited to compartments of the gluteal region, posterior tibial, thigh, forearm, hand and foot. Pressure builds in the compartment due to swelling from injury eventually exceeding capillary fill pressure. This causes impaired perfusion and ischemia, ultimately resulting in necrosis of multiple tissues, including muscle and nerve.

Diagnosing limbs at risk for compartment syndrome before the occurrence of irreversible damage to muscle and nerve is critical. Standard approaches for the diagnosis of impending compartment syndrome require clinical evaluation of the limb, including assessment for “tightness” of the overlying skin, reduced pulses distally and the degree of pain. However, such clinical assessment approaches are fairly non-specific and insensitive; compartment syndrome remains a clinical diagnosis made on the basis of pain, swelling, and the clinical judgment of individual providers. One of the few agreed-upon objective measurements of compartment syndrome requires the placement of a needle-pressure gauge, such as the C2Dx Intra-compartment Pressure Monitor System (Critical Care Diagnostics, Schoolcraft, MI, USA), to confirm elevated intra-compartmental pressures (generally above 30-45 mm Hg). Interestingly, this measures pressure, not tissue perfusion, and only indirectly provides information about the viability of tissue within the affected compartment. Difficulty with obtaining and interpreting invasive compartment pressure measurements has limited the clinical usefulness of these measurements, where they are only commonly used in situations where obtunded patients cannot provide an accurate clinical picture of evolving compartment syndrome and typically remain a single point-in-time measurement. While clinical judgment remains the gold standard for diagnosing compartment syndrome, inexperienced providers or those unfamiliar with compartment syndrome may not even consider the diagnosis of the condition until after ischemia and irreversible damage has already occurred.

Other approaches have also been suggested, including those based on alterations in ultrasound echo intensity and pO2 measured by near-infrared spectroscopy. However, all such approaches suffer from substantial limitations in that they need to be applied in the emergency room by physicians and that they cannot be readily used to assess patients over time. Indeed, many patients with leg injuries develop compartment syndrome in an insidious fashion with ACS occurring in as little as 12 hours, so a single measurement of pressure, ultrasound echo intensity, pO2 can miss subsequent onset of ACS which could occur as quickly as 12 hours after the single measurement. Also, ACS might be easily obscured by opioid and other analgesic therapies.

Thus, a portable, wireless, non-invasive approach for continuous monitoring for the development of compartment syndrome has substantial clinical value.

One approach that has not been studied is via the application of electrical impedance myography (EIM). In EIM, a weak, high frequency, directionally-specific, alternating current which is non-detectible by the patient, is passed between one pair of electrodes (the current emitting electrodes) through a region of tissue and the resulting surface voltages over that region are measured via an additional pair (the voltage measuring electrodes) from which the impedance characteristics, including the resistance, reactance, and phase angle, are calculated. As an example, in accordance with embodiments of the disclosed technology, a weak current may be any current with a root mean square (RMS) of less than 1 milliampere (mA). As an example, in accordance with embodiments of the disclosed technology, a high frequency current may be a current with a frequency in the range of 1 kilohertz (kHz) to 10 megahertz (MHz). EIM has already shown substantial value in detecting a number of neuromuscular disorders including amyotrophic lateral sclerosis and muscular dystrophy. It has also been shown to be sensitive to acute injury, including muscle strain. Additionally, other forms of electrical bioimpedance have been shown to be highly sensitive to cardiac ischemia. Thus, it logically follows that EIM methods might be very sensitive to the development of compartment syndrome-another form of ischemic injury, this time impacting skeletal rather than cardiac muscle.

Therefore, here we demonstrate the ability of EIM to detect early muscle ischemia in an established murine model of compression-induced muscle injury. We demonstrate that EIM measurements are sufficiently sensitive to detect early changes in compression-induced murine muscle ischemia. “Early”, as used here and throughout the present application, refers to a time before which injuries due to muscle ischemia have become irreversible. The sensitivity of EIM measurements to early muscle ischemia in the murine model suggests that EIM measurements in humans would be similarly sensitive to detection of early muscle ischemia.

The technology and methodology taught here can also be used to detect and monitor chronic compartment syndrome. Chronic compartment syndrome (CCS) is an exercise-induced condition characterized by recurrent pain and disability. Symptoms subside when the offending activity (usually running) is stopped but return when the activity is resumed. CCS may be considered an uncommon though important cause of exercise-induced leg and/or foot pain. The methods disclosed herein may be performed repeatedly over a period of time, at times during which the offending activity occurs, in order to monitor CCS. In some embodiments, the offending activity may be running, and the compartment experiencing CCS may be one of the lower extremities. In such embodiments, the methods disclosed herein could be performed while the lower extremity experiencing CCS is aggressively exercised, in order to monitor the progression of CCS in the lower extremity.

In the following description, numerous specific details are set forth regarding the systems and methods of the disclosed subject matter and the environment in which such systems and methods may operate, etc., in order to provide a thorough understanding of the disclosed subject matter. In addition, it will be understood that the examples provided below are exemplary, and that it is contemplated that there are other systems and methods that are within the scope of the disclosed subject matter.

### MATERIALS AND METHODS

### Surgical Procedure to Introduce Ischemic Injury

The project was approved by the Institutional Animal Care and Use Committee (IACUC) at Beth Israel Deaconess Medical Center (BIDMC). Twenty male, adult (13-weeks-old) Sprague Dawley rats were used in this study (Charles River Laboratories, Boston, MA, USA). Fifteen animals were subjected to ischemic injury, and the other five animals were used as non-injury controls. All animals were housed in the animal facility at BIDMC and provided food and water ad libitum. The animals were allowed a minimum of 72 hours to acclimatize prior to receiving any intervention.

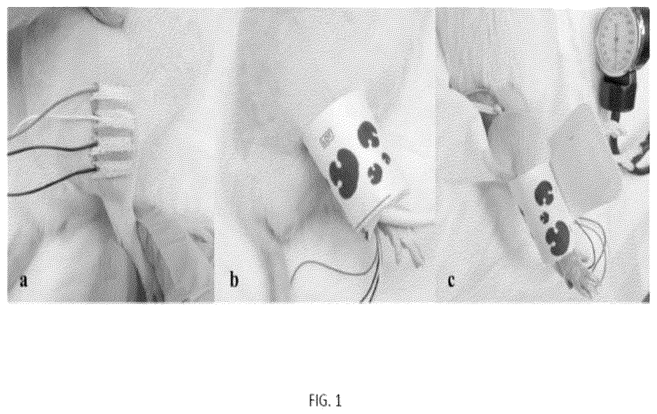

The animals were anaesthetized using isoflurane at 5% in an induction chamber, then maintained at 2% via a nose cone. After induction, rats were placed on a circulating water heating pad (Gaymar T/P® 500, Stryker, Kalamazoo, MI, USA) in left lateral decubitus position. They were then prepared by shaving the right hindlimb using a coarse-followed by fine-hair clippers. Extreme care was taken to prevent skin abrasions during the hair removal process. Four Ag/AgCl adhesive electrodes were cut to 2 mm wide sections using a razor blade. Spaced at 2 mm apart, the non-adhesive surfaces of the electrodes were attached to a 1×10 cm strip of adhesive tape. Sections of rubber were attached to the adhesive tape, filling the 2 mm gaps between the electrodes to act as an insulator (FIG. 1a). The anterolateral compartment of the leg was palpated between the knee and the ankle joints. After removal of the protective cover, the adhesive gel side of the electrodes was attached to the skin over the anterolateral compartment in a fashion perpendicular to the long axis of the leg. Once the appropriate positioning of the probes was confirmed, a size 3 neonatal blood pressure cuff was placed around the leg (FIG. 1b) and inflated to 300 mm Hg.

Hind limb ischemia was induced in 15 animals as described previously (ischemia group). Briefly, a 5 mm wide×1 mm thick rubber band was used as a tourniquet to induce arterial ischemia in the right hindlimb. The band was stretched and applied around the upper thigh as closely as possible to the groin and then tightened to maximum and double-knotted. Immediate paleness in the toes of the right hindlimb confirmed the ischemia. The tourniquet and the inflated pressure cuff were maintained for 40 minutes to ensure acute arterial ischemia (FIG. 1c).

### Electrical Impedance Myography Measurements

EIM measurements were performed with a Myolex® mView system (Myolex, Boston, MA, USA), using a previously described animal adaptor (20) [Must be included in this application]. Measurements were collected immediately after inflating the cuff (baseline), immediately after the tourniquet was applied (inflated tourniquet), followed by measurements every two minutes for the first ten minutes, every five minutes for the following 20 minutes, and finally once at the end of forty minutes of ischemic injury. At the end of the experiment, the rats were immediately euthanized using cardiac puncture while under anesthesia.

For the five non-injury control animals (control group), the same procedures for placement of the EIM electrodes were used without inducing ischemia. EIM measurements were made at non-injury baseline (control) by inflating the blood pressure cuff to 30 mm Hg to provide consistent contact between the electrodes and the animal skin.

A set of five measurements was collected for each time point described above. Resistance (R, Ω) and reactance (X, Ω) values, the outputs from the device, and calculated from the measured voltage characteristics, were collected over a frequency range of 10 kHz to 1 MHz.

Rather than using absolute impedance values, which can vary to some extent depending on the exact interelectrode distances, the size of the animal, and limb position, we chose to compare changes over time relative to baseline. Since both R and X are anticipated were expected to increase over time in relationship to the degree of muscle ischemia and injury, based on previous cardiac ischemia work, we developed the dimensionless relative injury index (RII), a simple product of the resistance and reactance values divided by the product of the baseline values, to help further amplify the impact of ischemic change.

### Immunohistochemistry

To validate the occurrence of tissue ischemia by our model, a subset of the animals (n=6) received pimonidazole HCl (Hypoxyprobe™-1, HPI, Inc., Burlington, MA, USA), injected intraperitoneally at a dose of 100 mg/kg, 90 minutes before initiation of ischemia. Pimonidazole HCl is distributed to all tissues via the systemic circulation and forms irreversible bonds
with both intracellular and extracellular proteins under hypoxic conditions. At the end of the experiment and before performing cardiac puncture, the muscles of the anterolateral and posterior compartment of the ischemic and contralateral sides were harvested. Muscles were immediately fixed in 4% paraformaldehyde. Twenty micron thick paraffin embedded sections were then processed and stained using HP-Red549-Kit (Hypoxyprobe™-1, HPI, Inc., Burlington, MA, USA) per manufacturer's instructions.

### Data Analysis

The Shapiro-Wilk test for normality was used to evaluate the distribution of the data. A repeated measures one-way analysis of variance (ANOVA) with Bonferroni confidence interval adjustment was performed to assess changes in reactance, resistance and RII over time.

Estimated marginal means was used to assess within-subject pairwise comparisons. Student's T-test was used to assess the difference between control and ischemia groups. The SPSS software (IBM-SPSS, version 26.0, Armonk, NY, USA) was used for data analysis. All comparisons were two-tailed, and a p value less than 0.05 was considered statistically significant.

### RESULTS

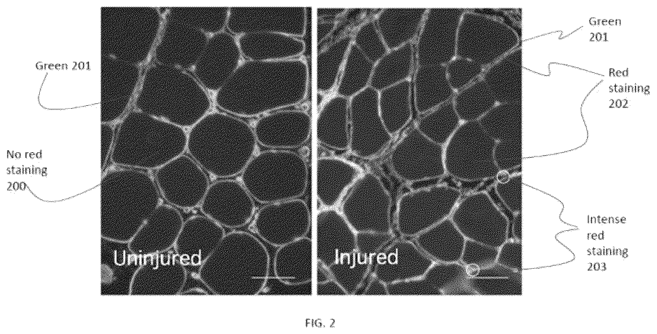

Hypoxyprobe™-1 staining demonstrated evidence of ischemia impacting both the muscle-associated vasculature (Intense red staining 203) as well as the myocytes (diffuse red staining 202) in the limb of a representative image from an animal experiencing induced-ischemia (FIG. 2, right panel) while the contralateral injured limb from the same animal showed no evidence of hypoxia (no red staining 200) (FIG. 2, left panel). These results support our use of this model as a reasonable proxy for the onset of ACS in rats to test the proposed EIM technology.

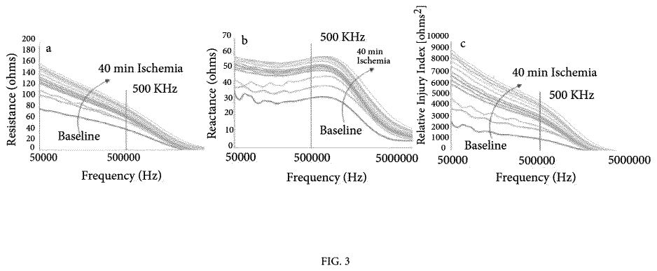

We assessed the sensitivity of EIM to ischemia development. FIGS. 3a, 3b, and 3c display the multi-frequency EIM data, with the x-axis of each figure representing frequency in Hertz (Hz). The y-axis of FIG. 3a represents resistance in Ohms, the y-axis of FIG. 3b represents reactance in Ohms, and the y-axis of FIG. 3c represents the Relative Injury Index (RII) in Ohms squared. We observed alterations in the multi-frequency EIM data, including elevations in both resistance and reactance values shown in FIGS. 3a and 3b respectively (data from a representative animal). The most dramatic changes occurred within just minutes of initiating the blood pressure cuff inflation and continued to develop out to over 40 minutes. Changes were present across the frequency spectrum.

Critically, these elevations in both impedance parameters were consistent with previous cardiac work cited above and predicted by impedance theory.

While resistance and reactance values both showed similar changes, combining the two measures and creation of an “ischemia injury index” provided an even more consistent measure of ischemic change over time, as shown in the multifrequency plot in FIG. 3c (note wider separation of values). Since both resistance and reactance measure different aspects of tissue health, this approach helps to ensure the greatest sensitivity to ischemic change.

While multifrequency data are interesting to demonstrate in graphical form, in order to develop a simpler monitoring mechanism, it will be useful to focus on just a single frequency or a limited set of frequencies. To that end, we compared a number of different individual frequencies; while many would be usable, the 500 kHz value appeared the most reliable across individual animals (data not shown) and thus was used in additional analyses.

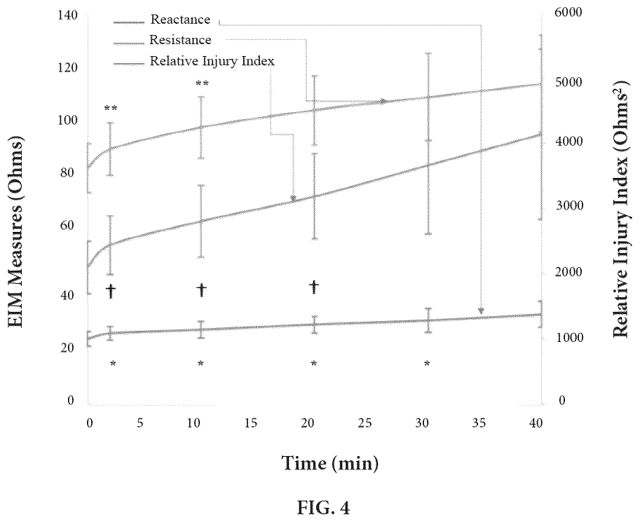

FIG. 4 presents the 500 kHz reactance, resistance and RII values averaged across all animals over the 40 minute ischemia injury period (measurements for 2, 10, 20, 30 and 40 minutes shown here only). The x-axis represents the time in minutes, the left y-axis represents the EIM measures of both reactance and resistance in ohms, and the right y-axis represents the measure of the RII in squared Ohms. Time 0 indicates the first measurement upon inflation of the tourniquet. Reactance, resistance and RII demonstrated significant changes over time (p=0.047, p=0.0005, and p=0.024 respectively).

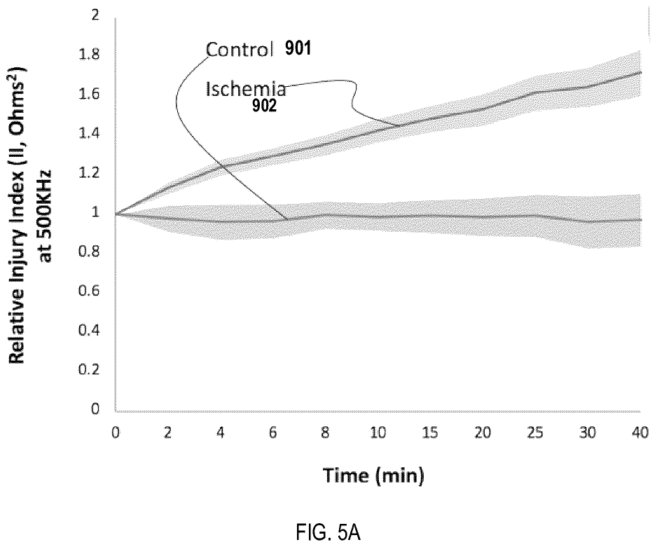

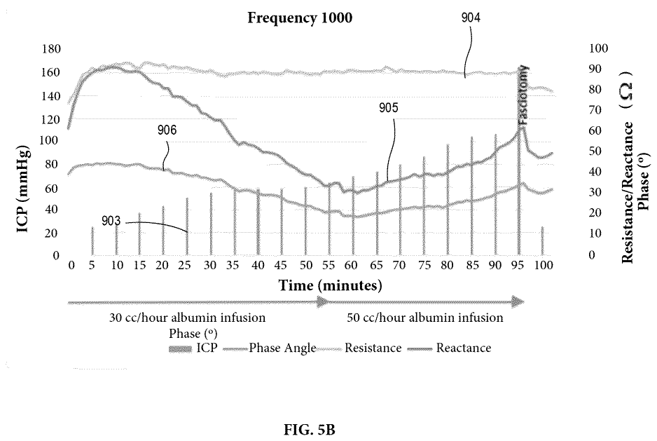

FIG. 5A shows Relative injury index (RII) over time in a group of animals (n=12) developing ischemia, shown as line 902, and a group of non-injury control animals (n=5) in which the blood pressure cuff was inflated to only 30 mmHg and no tourniquet was placed, shown as line 901. The x-axis represents the time in minutes, and the y-axis represents the RII in squared Ohms. The shaded areas around the lines represent values within a standard error of mean (positively and negatively). The figure demonstrates a significant difference between the control and ischemia groups for all time points from 2 minutes onward (p=0.0001). This further indicates that the observed changes are directly related to the induced injury in the limb.

FIG. 5B shows the change over time in resistance, reactance, phase angle, and intracompartmental pressure (ICP) measurements in a compartment of a single pig in which muscle ischemia was induced. The x-axis represents the time in minutes, the left y-axis represents the ICP in mmHg, and the right y-axis represents the resistance and reactance measured in ohms, as well as the phase measured in degrees. The frequency of the applied signal was 1000 Hz.

The bars 903 represent the ICP at various points in time. Over the time range of 0 to about 95 minutes on the x-axis, the ICP increases, as demonstrated by the increasing heights of bars 903. This increase reflects the fact that, during this time, the muscle in the compartment of the pig is infused with albumin (first at a rate of 30 cubic centimeters per hour, then at a rate of 50 cubic centimeters per hour). At about 95 minutes on the x-axis, a fasciotomy (involving cutting open the compartment via simple scalpel cuts into the tissue) is performed, leading to lowered pressure in the compartment. This lowered pressure is reflected in the sharp decrease in the heights of bars 903 after the fasciotomy is performed at about 95 minutes.

The measured resistance, shown as line 904, does not display significant change over time. Both the measured reactance, shown as line 905, and the measured phase, shown as line 906, display a clear decline over time initially. This decline reflects the infusion of albumin into the compartment, leading to increased free water in the compartment. However, at about 60 minutes on the x-axis, the measured reactance 905 and the measured phase 906 start to increase, reflecting that the infusion of albumin has caused the muscle to begin to become ischemic. This change in the measurements of reactance and phase is consistent with acute ischemia as observed in other applications (mainly cardiac ischemia). At the time of the fasciotomy at about 95 minutes, both reactance 905 and phase 906 begin to decrease, reflecting the lowered pressure in the compartment and beginning of reversal of the ischemia in the compartment.

### Device

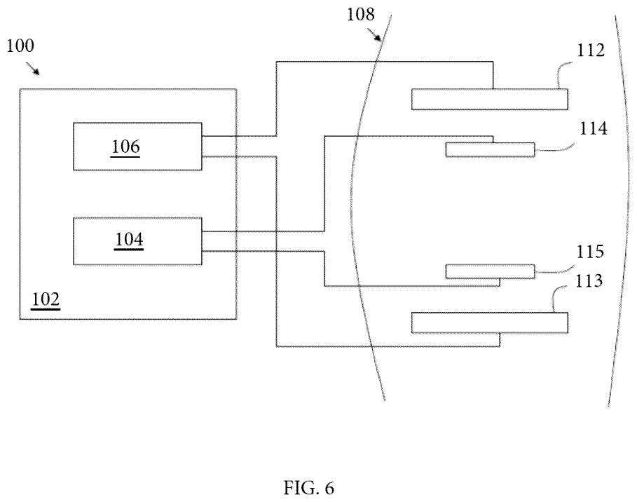

FIG. 6 illustrates an example of an apparatus 100 that may be used to perform multi-frequency EIM, according to some embodiments. Apparatus 100 includes electrodes 112-115, and also circuit 102 that measures and generates electrical signals using signal measurement circuit 104 and signal generation circuit 106. Apparatus 100 may include any components in any arrangement capable of delivering electrical signals and measuring electrical signals resulting from the electrical signals delivered, as the aspects of the disclosed subject matter are not limited in this respect.

In this embodiment, signal generating circuit 106 is coupled to two spaced-apart current-injecting electrodes 112 and 113, which may be applied to region of tissue 108. Using electrodes 112 and 113, an electrical signal is applied to region of tissue 108, for example, by passing an electrical current through the skin and into the region of tissue. The electrical signal that is applied may be any suitable signal, such as a predetermined voltage potential or a predetermined current. The electrodes may be isolated from a supply voltage using a transformer or any suitable device, such that a “floating” signal, and applied to the patient, thus enhancing the safety of the procedure.

In one embodiment, the signal that is applied to current-injecting electrodes 112 and 113 may be a sinusoidally varying current having a magnitude of no greater than 1 mA RMS (peak-to-peak) and a frequency between 1 kilohertz and 10 megahertz. As a consequence of applying this signal, electric current is injected into region of tissue 108. However, it should be appreciated that these values of voltage, shape and frequency are provided merely by way of illustration, as the disclosed subject matter is not limited in these respects. Furthermore, any suitable circuit and/or technique may be used to generate the electrical signal applied to the region of tissue, as the aspects of the disclosed subject matter are not limited for use with any particular method of electrical signal generation and/or application.

Signal measuring circuit 104 is coupled to two spaced-apart voltage-measuring electrodes 114 and 115. While the generated signal is applied to tissue region 108 by signal generation circuit 106, signal measurement circuit 104 measures a signal at the tissue region using voltage-measuring electrodes 114 and 115. The signal that is measured may be a voltage difference between the two electrodes that results from the generated signal. Any suitable circuit and/or technique may be used to measure the signal, as the aspects of the disclosed subject matter are not limited in this respect.

Circuit 102 may analyze the measured signal and determine a characteristic of the region of tissue based on the measured signal. Any suitable property of the signal may be measured, such as the magnitude, phase, impedance, resistance and reactance or any suitable combination thereof. In some embodiments, the measured voltage difference at electrodes 114 and 115 may be divided by the current applied through electrodes 112 and 113 to obtain an impedance measurement. Circuit 102 may determine an impedance, resistance, reactance, phase and/or any other suitable property of the region. Based on the measured signal, any of suitable electrical parameters, and/or electrical properties of the region of tissue, circuit 102 may determine a muscle characteristic. For example, circuit 102 may diagnose and/or assess a neuromuscular disease based on any suitable criteria, as discussed in further detail below.

In some circumstances, circuit 102 may display one or more of the determined electrical parameters to facilitate diagnosis and/or assessment by a physician or technician. Circuit 102 may include any suitable components for performing such measurements, calculations, determinations and presentation functions. As one example, circuit 102 may include a lock-in amplifier for impedance measurement, a computer for performing calculations and a display for displaying the results to a human (e.g., a technician or a physician). In some embodiments, the computer may store normative values based on larger population data for the purposes of analyzing impedance measurements. However, it should be appreciated that any suitable components or combination of components may be used, as the disclosed subject matter is not limited for use with any particular components or configuration of the components.

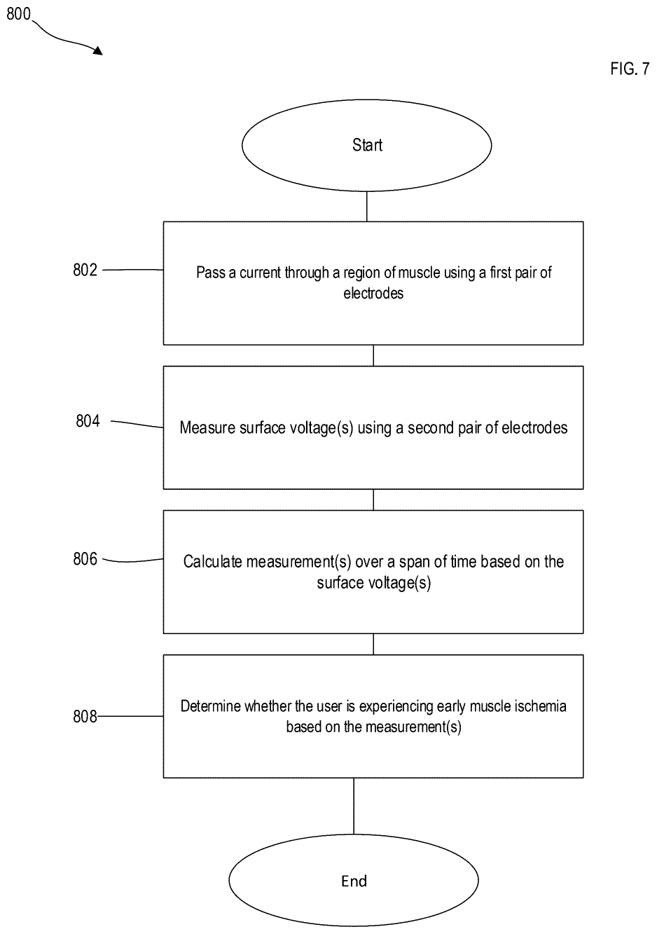

FIG. 7 is a flow chart illustrating a method 800 for detecting compartment syndrome, according to some embodiments. In step 802, a current is passed through a region of muscle of a user using a first pair electrodes. In the following step 804, a second pair of electrodes is used to measure one or more surface voltages that result from the passage of current through the region of muscle performed in step 802. In step 806, one or more measurements over a span of time are calculated based on the surface voltages measured in step 804. As described above, the one or more measurements calculated in step 806 may include an impedance, resistance, reactance, phase and/or any other suitable property of the region of muscle. In step 808, the one or more measurements calculated in step 806 are used to determine whether the user is experiencing early muscle ischemia. In some embodiments, the determination in step 808 of whether the user is experiencing early muscle ischemia may be based on both the measurements calculated in step 806, as well as a measurement of intracompartmental pressure using a needle-pressure gauge, as described above.

In some embodiments, the determination in step 808 may next be used to determine whether the user has compartment syndrome. If it is determined at step 808 that the user is experiencing early muscle ischemia, step 808 may be followed by a number of treatments, according to some embodiments. For example, in the case where the early muscle ischemia is a result of acute compartment syndrome, the early muscle ischemia may be treated by performing a surgical fasciotomy, which involves cutting open the compartment or muscle experiencing the early muscle ischemia by making cuts in the tissue of the compartment or muscle with a scalpel. As another example, in the case where the early muscle ischemia is a result of chronic compartment syndrome, the early muscle ischemia may be treated by instructing the user to cease engagement in an activity causing the early muscle ischemia.

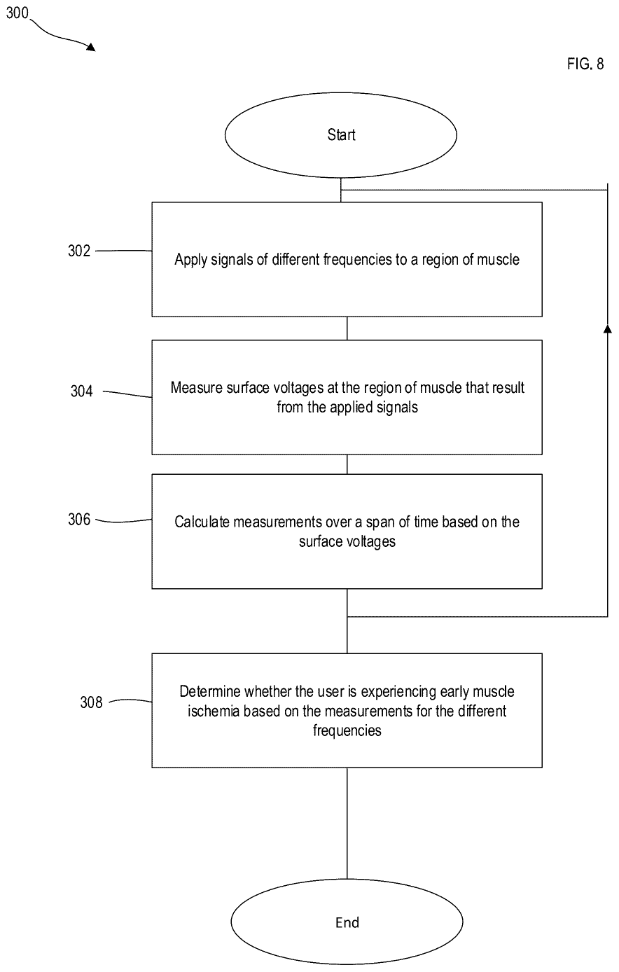

FIG. 8 is a flowchart of a method 300 for performing multi-frequency EIM, according to some embodiments. As described above, a first signal of a first frequency is applied to a tissue region in step 302, and a first surface voltage measurement is made in step 304, such that the measured surface voltage is a result of applying the first signal of the first frequency. In step 306, one or more measurements over a span of time are calculated based on the first surface voltage measurements corresponding to the first frequency.

Next, a second signal of a second frequency is applied to the tissue region in step 302, and a second surface voltage measurement is made for the second frequency in step 304. In step 306, one or more measurements over a span of time are calculated based on the second surface voltage measurements corresponding to the second frequency.

Further signals at different frequencies may also be applied, corresponding surface voltage measurements taken, and corresponding measurements over a span of time calculated. Any suitable number of frequencies may be used in the multi-frequency EIM procedure, as the disclosed subject matter is not limited as to the number of frequencies measured or the exact frequencies at which measurements are taken. Preferably, if multi-frequency EIM is performed, the frequencies used should be of a number and value such that the measurements are sufficient to provide information useful in assessment or diagnosis of the tissue region, e.g., the assessment or diagnosis of a muscle condition.

In step 308, it is determined whether the user is experiencing early muscle ischemia based on the measurements calculated for the different frequencies. This determination may be based upon the determination of a muscle characteristic, and/or one or more electrical properties obtained from the surface voltage measurements, such as the impedance, phase, resistance and/or reactance of the muscle. As another example, a frequency-averaged impedance, phase, resistance and or reactance may be determined for at least a portion of the range of frequency measurement. The frequency-averaged parameter may be a useful parameter for comparing healthy vs. unhealthy tissue, and evaluating changes in the tissue over time. For example, a diagnosis of a neuromuscular condition may be made based on a frequency-averaged parameter being above or below a threshold value.

One or more electrical properties obtained from measurements taken from the region of tissue as a function of frequency may be used as a signature for the region of tissue. The term signature refers herein to any collection of information obtained from a region of tissue that is characteristic of the tissue. The signature of the tissue, once obtained, may be analyzed to assess, diagnose or otherwise determine a characteristic and/or condition of the region of tissue.

The signature of the tissue may be computationally processed and/or analyzed or presented to a physician or technician for analysis. As one example, a plot of an electrical parameter vs. frequency (e.g., resistance, reactance or phase of the tissue vs. frequency) may be displayed on a computer monitor, and a physician may make a diagnosis based on the plot displayed. Multiple plots displaying any of various electrical properties of the tissue with respect to frequency may be displayed, as the aspects of the disclosed subject matter are not limited in this respect.

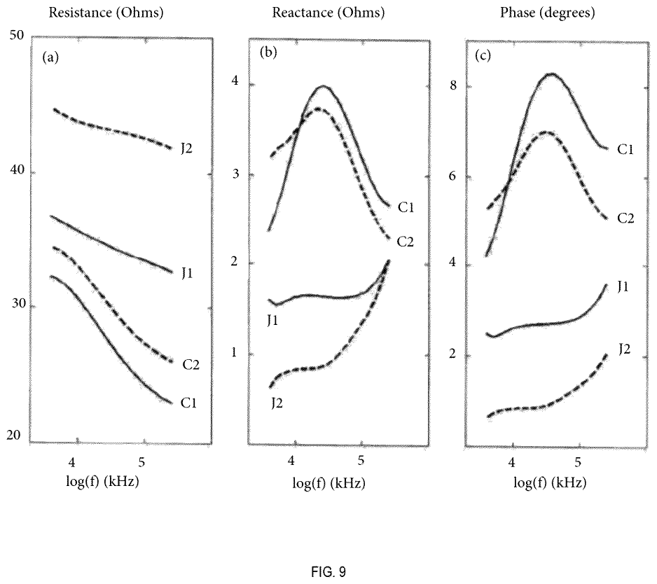

FIGS. 9A-C show plots of measured electrical parameters versus frequency, using multi-frequency electrical impedance myography obtained in accordance with various aspects of some embodiments. In particular, FIGS. 9A-C show plots of resistance, reactance and phase vs. the logarithm of frequency, respectively, measured for two different amyotrophic lateral sclerosis (ALS) patients (C and J) at two different visits, 3-4 months apart each. The x-axis in each of FIGS. 9A-C represents the logarithm of the frequency of the applied signal, where the frequency is in kilohertz (kHz). The y-axis of FIG. 9A represents the measured resistance in Ohms; the y-axis of FIG. 9B represents the measured reactance in Ohms, and the y-axis of FIG. 9C represents the measured phase in degrees. In each figure, the solid lines show the measurements taken at the first visits and the dashed lines show the measurements taken at the follow-up visits. Patient C had relatively mild ALS, while patient J had a more severe form of the disease. As can be readily appreciated from FIGS. 9B-C, the patient (J) with the more severe muscle disorder had lower phase and reactance values than the patient (C) with the less severe disorder. At the follow-up visit, both patients exhibited primarily a decrease in both phase and reactance measured, which illustrates the progression of the disease over time.

Additionally, the patient with less severe ALS had a much more pronounced frequency peak in both reactance and phase than the patient with the more severe form of ALS. These results are provided to illustrate various criteria that may be used in determining characteristics of a region of tissue using EIM, and which may be displayed for use by a suitable medical practitioner. However, it should be appreciated that any suitable measured and/or calculated criteria may be used for characterization, as the disclosed subject matter is not limited as to the particular criteria used. In some circumstances, a diagnoses or assessment may be made by circuit 102. For example, circuit 102 may be configured to analyze the signature (e.g., one or more electrical properties as a function of frequency) to determine a characteristic of the region of tissue and/or to assess a condition of the region of tissue.

As discussed above, multidirectional (or rotational) EIM may be used for the assessment and characterization of a region of tissue. Multidirectional EIM can be performed by measuring the voltage difference between voltage-measuring electrodes that are arranged with a desired orientation with respect to an axis of the muscle fibers. Both the current-injecting electrodes and the voltage-recording electrodes may have the same orientation with respect to the muscle fibers. The electrical properties of the region of tissue at various orientations may be used to characterize the region of tissue, e.g., to assess a condition of the muscle and/or to perform a diagnosis of the muscle.

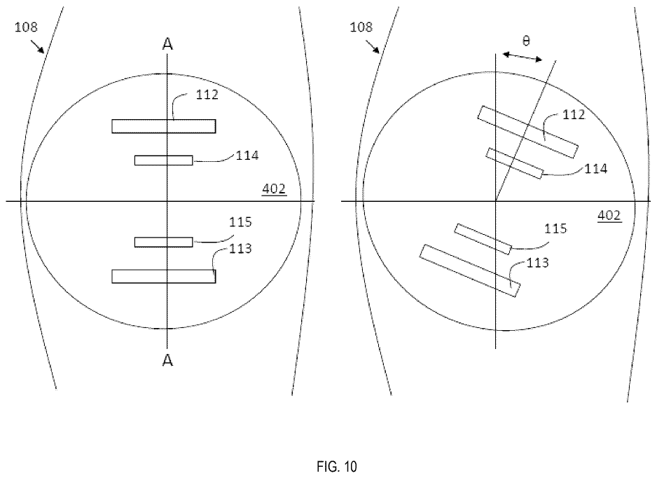

FIG. 10 is a diagram illustrating performing EIM at different orientations with respect to a region of tissue 108. FIG. 10 shows electrodes 112-115, as described above with respect to FIG. 6. Electrodes 112-115 may be mounted on a base 402. The left panel of FIG. 10 illustrates performing EIM along a direction A-A aligned with an axis of the region of tissue 108, e.g., substantially aligned with fibers of the muscle. The right panel of FIG. 10 illustrates performing EIM along a direction at an angle θ with respect to the axis. Measurements obtained at the different orientations may be used to characterize and/or otherwise assess a condition of the region of tissue 108. Measurements may be obtained at multiple orientations to obtain information about how properties of the tissue vary with orientation (e.g., to determine a degree of anisotropy of the tissue), as discussed in further detail below.

In one embodiment, electrodes 112-115 are mounted on rotatable base 402, which is made of electrically insulating material. When a measurement is to be taken, electrodes 112-115 are brought into contact with the skin at the region of tissue, and are aligned in a first direction with respect to an axis of the region. When a second measurement is to be taken, base 402 is rotated by the desired angle θ, and electrodes 112-115 are again brought into contact with the skin at the new orientation.

Multiple different measurements may be made at different angles. As one example, measurements may be made at six different angles, each 30° apart (0°, 30°, 60°, 90°, 120° and 150°). However, it should be appreciated that any suitable angle increments or number of measurements at different angles may be used, as the disclosed subject matter is not limited in this respect. Preferably, if rotational EIM is performed, the angles used should be of a number and increment such that the measurements are sufficient to provide information useful in assessment or diagnosis of the tissue region, e.g., the assessment or diagnosis of a muscle condition.

In another embodiment, electrodes 112-115 may be mounted on rotatable base 402 made of an electrically conductive material. In this embodiment, the electrically conductive base 402 may be brought into contact with the skin at the region of measurement, and the electrodes themselves may not contact the region directly. In one example, electrically conductive base 402 may be anisotropically conductive such that it preferentially conducts current in a direction perpendicular to the base (e.g., into the patient's body). The anisotropy of the conductive base can prevent undesirable cross-talk between the electrodes, and may allow current to penetrate a greater depth into the tissue region.

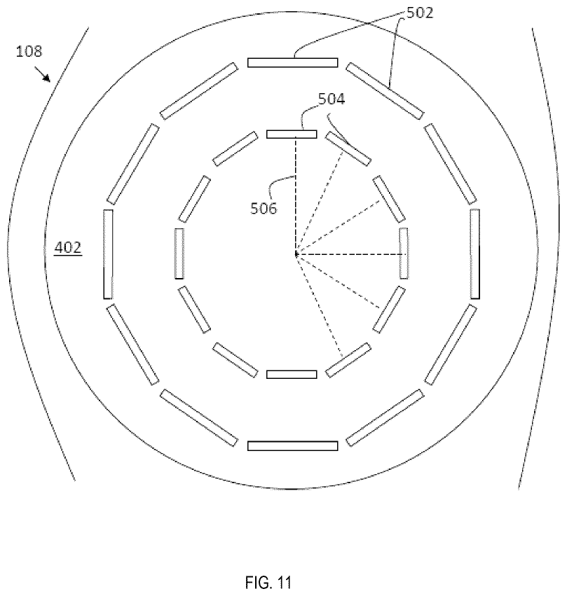

FIG. 11 illustrates another embodiment using rotational EIM, in which a plurality of current-injecting electrodes 502 and voltage-measuring electrodes 504 are mounted on base 402 at different orientations. Since the electrodes are mounted at a plurality of different orientations, it may not be necessary to rotate the electrodes or base 402 to make measurements at different angles. When a first measurement is to be made, an appropriate pair of current-injecting electrodes can be selected and coupled to signal-generating circuit 106 using any suitable switches. That is, the plurality of electrodes may be configured such that the combination of electrodes 502 and 504 at any desired orientation may be selectively activated. For example, the current-injecting electrodes that lie along line 506 may be selected first.

Additionally, the appropriate pair of voltage measuring electrodes 504 that lie along line 506 may be selected, and may be coupled to signal-measuring circuit 104 using any suitable switches. A first measurement may then be taken along direction 506. When a measurement is to be made along a different direction, the switches may be reconfigured to couple different electrodes 502 and 504 to the appropriate circuits, and measurement may be taken at a different orientation.

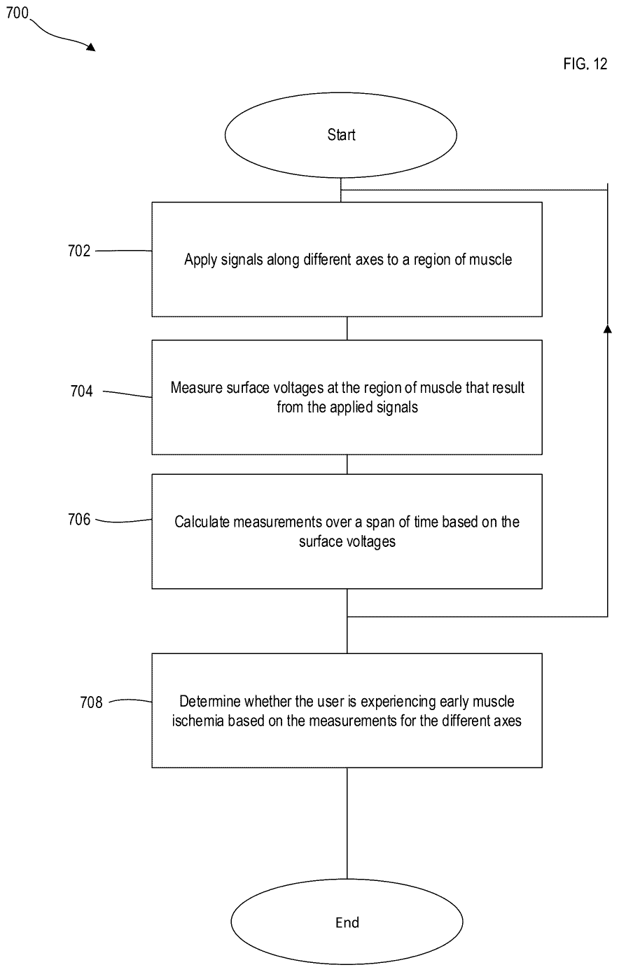

FIG. 12 is a flow chart of a method 700 of performing rotational EIM, according to some embodiments. In step 702, a first signal is applied to a tissue region at a first orientation, and in step 704 a surface voltage, resulting from the first applied signal, is measured at the region. In step 706, one or more measurements over a span of time are calculated based on the surface voltages measured in step 704.

Next, a second signal is applied to the tissue region at a different orientation. For example, a linear electrode array may be rotated, and another measurement may be made, as illustrated in FIG. 10. As another example, if a quasi-circular electrode array is used (FIG. 11), a different set of electrodes may be selected that correspond to a different orientation, and a corresponding measurement may be made. It is preferred that at least one measurement be made along a muscle axis, and that at least one measurement be made perpendicular to the muscle axis. Finally, in step 708, it is determined whether the user is experiencing early muscle ischemia based on the measurements calculated at the different orientations. This determination may be based upon the determination of a muscle characteristic, and/or one or more electrical properties obtained from the surface voltage measurements, such as the impedance, phase, resistance and/or reactance of the muscle.

The one or more electrical properties obtained as a function of orientation may be used as a signature of the region of tissue. As discussed above, this signature may be analyzed to determine a characteristic of the muscle and/or to assess a condition of the muscle. For example, how the one or more electrical properties vary with orientation (e.g., a degree of anisotropy) may be used to assess the health of the tissue and/or diagnose a condition such as a specific neuromuscular disorder. The signature may be analyzed quantitatively, or compared to a reference signature obtained from known healthy or diseased tissue to assist in the analysis and/or diagnosis of the tissue.

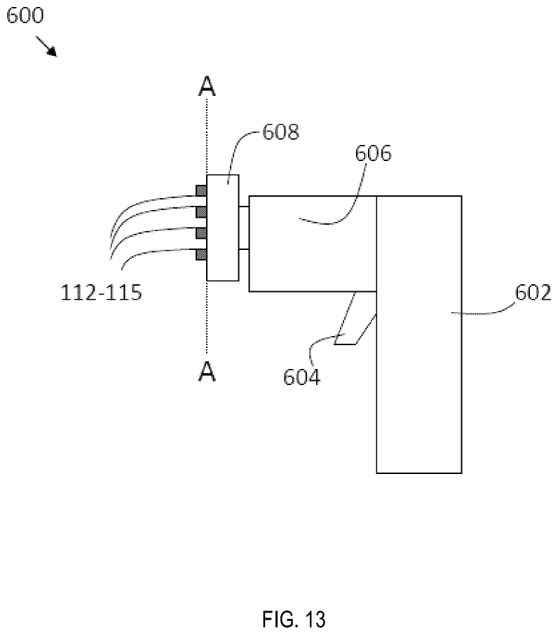

FIG. 13 illustrates an example of a hand-held apparatus 600 that may be used for performing EIM, including rotational and/or multi-frequency EIM. Providing a hand-held EIM device may facilitate making EIM measurements, and thus may reduce the amount of time needed to make the measurements. Hand-held apparatus 600 may include a handle 602, a user interface 604, a body 606, base 608 and electrodes 112-115. The electrodes may be coupled to circuit 102 in any suitable way, such as through a cord attached at the bottom of handle 602, for example. FIG. 13 illustrates direction A-A corresponding to direction A-A illustrated in the left panel of FIG. 10.

In one embodiment, base 608 may be rotatable, as discussed above, for performing rotational EIM. In another embodiment, base 608 may not be rotatable, but may have a plurality of electrodes 502 and 504 positioned at different orientations, as described in connection with FIG. 11. Apparatus 600 may be configured such that either technique may be used, depending on the type of base/electrode combination that is mounted to the apparatus. In some circumstances, it may be desirable to provide multiple different base/electrode combinations of different sizes that may be easily interchangeable for measuring different types of muscles, or muscles of different sizes. When a different size is needed, the base 608 may be detached from apparatus 600 and another base may be attached.

### Illustrative Computer

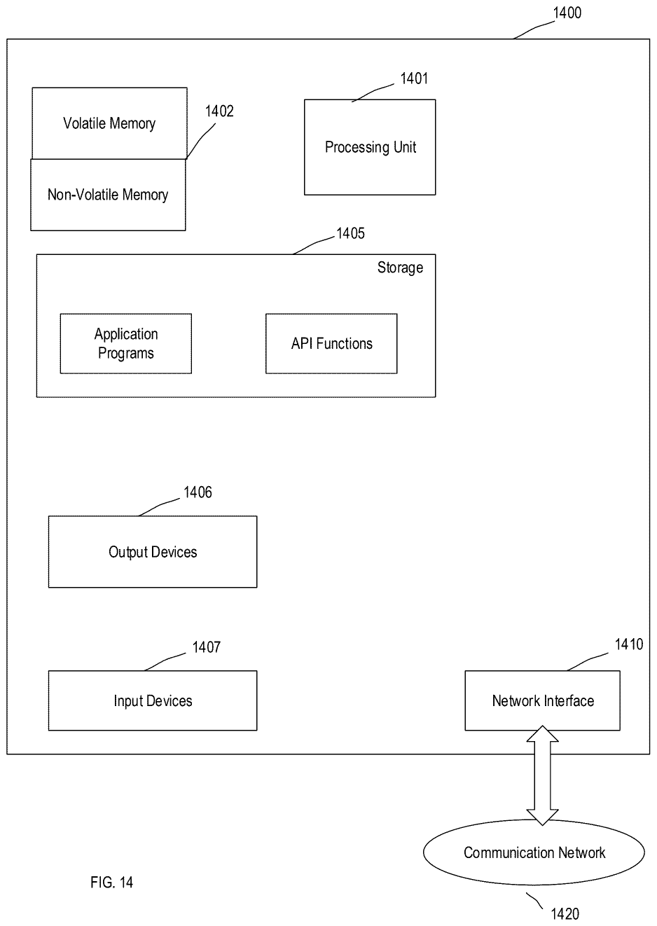

FIG. 14 shows, schematically, an illustrative computer 1400 on which any aspect of the present disclosure may be implemented.

In the embodiment shown in FIG. 14, the computer 1400 includes a processing unit 1401 having one or more processors and a non-transitory computer-readable storage medium 1402 that may include, for example, volatile and/or non-volatile memory. The memory 1402 may store one or more instructions to program the processing unit 1401 to perform any of the functions described herein. The computer 1400 may also include other types of non-transitory computer-readable medium, such as storage 1405 (e.g., one or more disk drives) in addition to the system memory 1402. The storage 1405 may also store one or more application programs and/or resources used by application programs (e.g., software libraries), which may be loaded into the memory 1402.

The computer 1400 may have one or more input devices and/or output devices, such as devices 1406 and 1407 illustrated in FIG. 14. These devices may be used, for instance, to present a user interface. Examples of output devices that may be used to provide a user interface include printers and display screens for visual presentation of output, and speakers and other sound-generating devices for audible presentation of output. Examples of input devices that may be used for a user interface include keyboards and pointing devices (e.g., mice, touch pads, and digitizing tablets). As another example, the input devices 1407 may include a microphone for capturing audio signals, and the output devices 1406 may include a display screen for visually rendering, and/or a speaker for audibly rendering, recognized text.

In the example shown in FIG. 14, the computer 1400 also includes one or more network interfaces (e.g., the network interface 1410) to enable communication via various networks (e.g., the network 1420). Examples of networks include a local area network (e.g., an enterprise network) and a wide area network (e.g., the Internet). Such networks may be based on any suitable technology and operate according to any suitable protocol, and may include wireless networks and/or wired networks (e.g., fiber optic networks).

### DISCUSSION

The diagnosis of compartment syndrome remains a challenge. Overlooking it entirely or diagnosing it late has major negative clinical implications. It is often referred to as a “clinical diagnosis,” in that physicians must balance a range of factors including pain, physical exam findings, and either subjective estimations or objective measurements of compartment pressure to determine whether compartment syndrome is present or impending. Yet the one crucial physiological feature—inadequate tissue perfusion—is only indirectly evaluated by these approaches, including direct compartment pressures. In this study, we hypothesized that EIM could provide a better direct indicator of muscle hypoperfusion.

In EIM, a weak current is passed between electrodes, and the resultant voltage measurements can reflect the condition of the muscle through which this current passes. As an example, a weak current may be any current with a root mean square of less than 1 mA. A number of tissue features contribute to the measured impedance in a complex fashion. Among these include the amount of free intracellular and extracellular water, membrane integrity, ion concentrations, and capillary blood flow. Reduced tissue perfusion would be expected to have a multitude of effects, including reducing free water, inhibiting blood flow, and causing membrane instability. Cumulatively, these have the impact of increasing the measured reactance and resistance values. Thus, in some sense, the impedance-based alterations observed here reflect a more appropriate measure of perfusion than the clinical measures and compartmental pressures currently utilized.

The experimental model used to test our hypothesis appeared to generate reproducible hypoxia as confirmed by the Hypoxyprobe™ staining of both vascular endothelial cells (intense red staining 203) and myocytes (diffuse red staining 202) within the muscle from the limb undergoing study; no such positive staining was evident in the muscle excised from the contralateral uninjured limb. Under conditions of ischemia, we were able to demonstrate a significant escalation in both resistance and reactance values during the 40-minute period of ischemia. This increase in both resistance and reactance was absent when measurements were collected from the healthy animals not subjected to ischemic injury. As such, EIM signal showed both reproducibility and stability under normal circumstance. Observed changes in impedance could be confidently attributed to tissue ischemia within the measured compartment. The sensitivity of the EIM measurements performed on animals to detection of early muscle ischemia suggests that similar EIM measurements performed on humans would also be sufficiently sensitive to the detection of early muscle ischemia.

It is important to note that ischemia from clinical ACS or CCS occurs on a continuum, and the degree of permanent muscle damage is related to the time course of exposure to hypoxia. Early in its time course, muscle changes from ACS or CCS may be partially or wholly reversible, while prolonged exposure can be sufficiently severe to lead to irreversible damage. Our data also demonstrates a dose response to increasing time of exposure to hypoxia. While both resistance and reactance show this dose response, our data suggests that measurements obtained by combining both measures into a single measure provides greater sensitivity to early injury. With increasing time of exposure to hypoxia, the RII increases, demonstrating the most rapid increases during the earliest time points. These differences are particularly obvious in relation to the controls. Since these measurements can be made noninvasively, the contralateral limb could be potentially used as a simple internal control for any given patient suspected of potentially developing compartment syndrome. Further, with this approach, control measurements are unlikely to be influenced by the mean arterial pressure in the same manner that compartment pressures can be affected, as the measurements are a more direct means of assessing tissue hypoperfusion and resultant hypoxia.

The data may be collected frequently, potentially even in the presence of a cast (via electrodes placed within the cast itself prior to its placement), brace, wrapping, or bandage. This could help improve our ability to detect subtle but significant changes indicative of developing ACS or CCS.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1. A method for detecting compartment syndrome, the method comprising:
passing, using a first pair of electrodes, a current through a region of muscle of a patient user;
measuring, using a second pair of electrodes, one or more surface voltages;
calculating, based on the one or more surface voltages, one or more measurements over a span of time; and
determining, based on the one or more measurements over the span of time, whether the muscle being measured is experiencing early muscle injury.

2. The method of claim 1, wherein the determining, based on the one or more measurements over the span of time, whether the patient is experiencing early muscle injury comprises:
comparing the one or more measurements to threshold values.

3-10. (canceled)

11. The method of claim 1, further comprising determining whether the patient has compartment syndrome.

12. The method of claim 1, wherein the first pair of electrodes and the second pair of electrodes are positioned beneath the surface of a cast.

13. The method of claim 1, wherein:
passing, using the first pair of electrodes, a current through a region of muscle of a patient comprises varying a frequency of an alternating current over a plurality of frequency values;
measuring, using the second pair of electrodes, one or more surface voltages comprises measuring a plurality of voltages from the second pair of electrodes, each voltage of the plurality of voltages corresponding to a frequency value of the plurality of frequency values;
calculating, based on the one or more surface voltages, one or more measurements over a span of time comprises calculating a plurality of measurements, each measurement of the plurality of measurements corresponding to a voltage of the plurality of voltages; and
determining, based on the one or more measurements over the span of time, whether the patient is experiencing early muscle injury comprises determining whether the patient is experiencing early muscle injury based on the plurality of frequency values and the plurality of measurements.

14. The method of claim 13, wherein determining whether the patient is experiencing early muscle injury based on the plurality of frequency values and the plurality of measurements comprises determining a frequency-averaged parameter for at least a portion of the plurality of frequency values.

15. The method of claim 14, wherein determining whether the patient is experiencing early muscle injury based on the plurality of frequency values and the plurality of measurements comprises determining whether the frequency-averaged parameter exceeds a threshold value.

16. The method of claim 1, wherein:
passing, using a first pair of electrodes, a current through a region of muscle of a patient comprises passing a current through a region of muscle of a patient along each axis of a plurality of axes;
measuring, using the second pair of electrodes, one or more surface voltages comprises measuring a plurality of voltages from the second pair of electrodes, each voltage of the plurality of voltages corresponding to an axis of the plurality of axes;
calculating, based on the one or more surface voltages, one or more measurements over a span of time comprises calculating a plurality of measurements, each measurement of the plurality of measurements corresponding to a voltage of the plurality of voltages; and
determining, based on the one or more measurements over the span of time, whether the patient is experiencing early muscle injury comprises determining whether the patient is experiencing early muscle injury based on the plurality of axes and the plurality of measurements.

17. The method of claim 1, further comprising obtaining a pressure measurement for the region of muscle, and wherein:
determining, based on the one or more measurements over the span of time, whether the user is experiencing early muscle injury comprises determining, based on the one or more measurements over the span of time and the pressure measurement, whether the user is experiencing early muscle ischemia.

18. The method of claim 1, further comprising treating the early muscle injury by releasing pressure in the region of muscle by creating an incision into the region of muscle.

19. (canceled)

20. A system for detecting compartment syndrome, the system comprising:
a first pair of electrodes;
a second pair of electrodes; and
at least one computing device communicatively coupled to the first pair of electrodes and the second pair of electrodes, the at least one computing device comprising a computer processor configured to perform the acts of:
passing, using the first pair of electrodes, a current through a region of muscle of a patient;
measuring, using the second pair of electrodes, one or more surface voltages;
calculating, based on the one or more surface voltages, one or more measurements over a span of time; and
determining, based on the one or more measurements over the span of time, whether the patient is experiencing early muscle injury

21-29. (canceled)

30. The system of claim 20, wherein the computing device processor is further configured to perform the act of determining whether the patient has compartment syndrome.

31. The system of claim 20, wherein the first pair of electrodes and the second pair of electrodes are positioned beneath the surface of a cast.

32. The system of claim 20, wherein:
passing, using the first pair of electrodes, a current through a region of muscle of a patient comprises varying a frequency of an alternating current over a plurality of frequency values;
measuring, using the second pair of electrodes, one or more surface voltages comprises measuring a plurality of voltages from the second pair of electrodes, each voltage of the plurality of voltages corresponding to a frequency value of the plurality of frequency values;
calculating, based on the one or more surface voltages, one or more measurements over a span of time comprises calculating a plurality of measurements, each measurement of the plurality of measurements corresponding to a voltage of the plurality of voltages; and
determining, based on the one or more measurements over the span of time, whether the patient is experiencing early muscle injury comprises determining whether the patient is experiencing early muscle injury based on the plurality of frequency values and the plurality of measurements.

33. The system of claim 32, wherein determining whether the patient is experiencing early muscle injury based on the plurality of frequency values and the plurality of measurements comprises determining a frequency-averaged parameter for at least a portion of the plurality of frequency values.

34. The system of claim 33, wherein determining whether the patient is experiencing early muscle injury based on the plurality of frequency values and the plurality of measurements comprises determining whether the frequency-averaged parameter exceeds a threshold value.

35. The system of claim 20, wherein:
passing, using a first pair of electrodes, a current through a region of muscle of a patient user comprises passing a current through a region of muscle of a patient along each axis of a plurality of axes;
measuring, using the second pair of electrodes, one or more surface voltages comprises measuring a plurality of voltages from the second pair of electrodes, each voltage of the plurality of voltages corresponding to an axis of the plurality of axes;
calculating, based on the one or more surface voltages, one or more measurements over a span of time comprises calculating a plurality of measurements, each measurement of the plurality of measurements corresponding to a voltage of the plurality of voltages; and
determining, based on the one or more measurements over the span of time, whether the patient is experiencing early muscle injury comprises determining whether the patient is experiencing early muscle injury based on the plurality of axes and the plurality of measurements.

36. The system of claim 20, further comprising a hand-held apparatus, wherein:
the first pair of electrodes and the second pair of electrodes are mounted on the hand-held apparatus; and
the first pair of electrodes and the second pair of electrodes are communicatively coupled to the at least one computing device by means of the hand-held apparatus.

37. At least one non-transitory computer-readable storage medium storing processor-executable instructions that, when executed by at least one computer hardware processor, cause the at least one computer hardware processor to perform:
passing, using a first pair of electrodes, a current through a region of muscle of a patient;
measuring, using a second pair of electrodes, one or more surface voltages;
calculating, based on the one or more surface voltages, one or more measurements over a span of time; and
determining, based on the one or more measurements over the span of time, whether the patient is experiencing early muscle injury.

38-51. (canceled)

52. The at least one non-transitory computer-readable storage medium of claim 37, wherein:
passing, using a first pair of electrodes, a current through a region of muscle of a patient comprises passing a current through a region of muscle of a patient along each axis of a plurality of axes;
measuring, using the second pair of electrodes, one or more surface voltages comprises measuring a plurality of voltages from the second pair of electrodes, each voltage of the plurality of voltages corresponding to an axis of the plurality of axes;
calculating, based on the one or more surface voltages, one or more measurements over a span of time comprises calculating a plurality of measurements, each measurement of the plurality of measurements corresponding to a voltage of the plurality of voltages; and
determining, based on the one or more measurements over the span of time, whether the patient is experiencing early muscle injury comprises determining whether the patient is experiencing early muscle injury based on the plurality of axes and the plurality of measurements.
